(19) United States
(12) Patent Application Publication
(10) Pub. No.: US 2010/0317831 A1
(43) Pub. Date: Dec. 16, 2010

(54) PROBE COMPOUNDS FOR PROTEIN TYROSINE PHOSPHATASE (PTP) AND PRECURSORS THEREOF

(75) Inventor: Lee-Chiang Lo, Taipei (TW)

Correspondence Address: PAI PATENT & TRADEMARK LAW FIRM, 1001 FOURTH AVENUE, SUITE 3200, SEATTLE, WA, 98154, US

(73) Assignee: NATIONAL TAIWAN UNIVERSITY, Taipei (TW)

(21) Appl. No.: 12/814,454

(22) Filed: Jun. 13, 2010

Related U.S. Application Data
(60) Provisional application No. 61/186,883, filed on Jun. 14, 2009.

(51) Int. Cl. C07K 5/083; C07F 9/12; C07F 9/6574

(52) U.S. Cl. 530/331; 558/190; 558/86

(57) ABSTRACT

A probe compound for protein tyrosine phosphatases (PTPs), as shown in Formula (I) is provided.

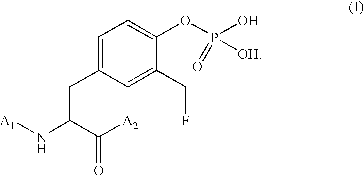

In Formula (I), A₁ and A₂ represent amino acids. The amino acids include leucine, phenylalanine, glutamic acid, lysine, alanine, arginine, aspartic acid, asparagine, citrulline, cysteine, cystine, glutamine, glycine, histidine, hydroxyproline, isoleucine, methionine, proline, serine, threonine, tryptophan, valine or a combination thereof. The invention also provides probe compound precursors for protein tyrosine phosphatases (PTPs).

### CROSS REFERENCE TO RELATED APPLICATIONS

This application claims the benefit of U.S. Provisional Application No. 61/186,883, filed Jun. 14, 2009, the entirety of which is incorporated by reference herein.

### BACKGROUND OF THE INVENTION

1. Field of the Invention

The invention relates to a probe compound, and more particularly to a probe compound for protein tyrosine phosphatases (PTPs) and a precursor thereof.

2. Description of the Related Art

Phosphatases play a crucial role in physiology, participating in cell growth, differentiation, metabolism and signal transduction. Currently, research in protein tyrosine phosphatase (PTP) subfamilies is popular.

A complete probe compound design comprises four parts. For example, a recognition unit, a trapping mechanism, a linker and a reporter group. When the recognition unit binds to an enzyme, a highly reactive intermediate is formed after enzymatic hydrolysis. The intermediate immediately forms a covalent bond with the enzyme. A probe compound-enzyme adduct is then detected and purified through the reporter group. Improvements in the specificity and selectivity between the probe compound and the enzyme, specifically, protein tyrosine phosphatases (PTPs), is desired.

### BRIEF SUMMARY OF THE INVENTION

One embodiment of the invention provides a probe compound for protein tyrosine phosphatases (PTPs), as shown in Formula (I):

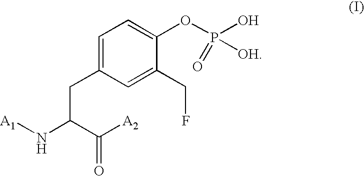

In Formula (I), A₁ and A₂ represent amino acids.

The amino acids comprise leucine, phenylalanine, glutamic acid, lysine, alanine, arginine, aspartic acid, asparagine, citrulline, cysteine, cystine, glutamine, glycine, histidine, hydroxyproline, isoleucine, methionine, proline, serine, threonine, tryptophan, valine or a combination thereof.

The probe compound further comprises a linker connected to A₁ or A₂. The linker comprises 2,2′-(ethylenedioxy)bis(ethylamine).

The probe compound further comprises a reporter group connected to the linker. The reporter group comprises biotin.

In the design of the disclosed probe compound, the fluoro-modified tyrosine phosphate is a core structure of a recognition unit. It comprises an ortho-fluoromethyl phosphotyrosine derivative. After hydrolysis, the recognition unit is converted to a highly reactive quinone methide intermediate through 1,4-elimination, which is capable of undergoing alkylation with suitable nucleophilic groups on protein tyrosine phosphatase (PTP). The enzyme specificity of the probe compound is improved by tuning the amino acid sequences (type and length) of the recognition unit. In addition to the fluorine atom, other halogen atoms, for example chlorine, bromine or iodine, are inappropriate for use due to direct reaction with the nucleophilic group of the enzyme, which would form a non-specific alkylation.

One embodiment of the invention provides a probe compound precursor for protein tyrosine phosphatases (PTPs), as shown in Formula (II):

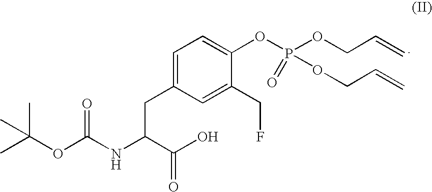

One embodiment of the invention provides a probe compound precursor for protein tyrosine phosphatases (PTPs), as shown in Formula (III):

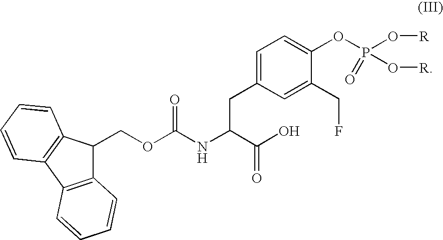

In Formula (III), R represents —CH₂CH═CH₂ or —CH₂C₆H₅.

The disclosed probe compound precursor of Formula (III) is suitable to be applied in Fmoc chemistry peptide synthesis, for example Fmoc chemistry solid phase peptide synthesis (SPPS). When R=-Bn, the peptide product is cut from a solid phase carrier by treatment with a trifluoroacetic acid (TFA) reagent, which simultaneously removes all the side chain protecting groups of the peptide, simplifying the synthesis processing steps.

One embodiment of the invention provides a probe compound for protein tyrosine phosphatases (PTPs), as shown in Formula (IV):

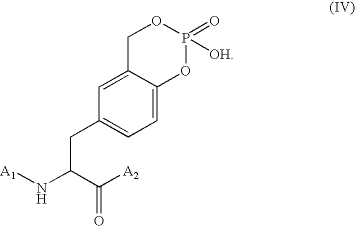

In Formula (IV), A₁ and A₂ represent amino acids.

The amino acids comprise leucine, phenylalanine, glutamic acid, lysine, alanine, arginine, aspartic acid, asparagine, citrulline, cysteine, cystine, glutamine, glycine, histidine, hydroxyproline, isoleucine, methionine, proline, serine, threonine, tryptophan, valine or a combination thereof.

The probe compound further comprises a linker connected to A₁ or A₂. The linker comprises 2,2′-(ethylenedioxy)bis(ethylamine).

The probe compound further comprises a reporter group connected to the linker. The reporter group comprises biotin.

One embodiment of the invention provides a probe compound precursor for protein tyrosine phosphatases (PTPs), as shown in Formula (V):

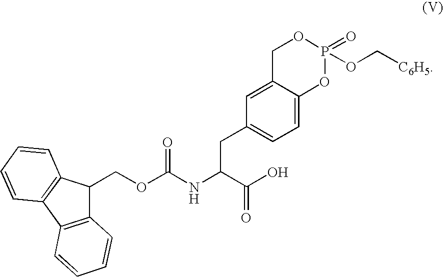

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIGS. 1a to 1d show the results of protein tyrosine phosphatase 1B (PTP1B) and T-cell protein tyrosine phosphatase (TCPTP) labeling of a probe compound according to one embodiment of the invention;

FIGS. 2a to 2c show the enzyme specificity of probe compounds for various enzymes according to one embodiment of the invention;

FIGS. 3a to 3d show the enzyme specificity of probe compounds for various enzymes according to one embodiment of the invention;

FIGS. 4a to 4e show the enzyme selectivity of probe compounds for various PTPs according to one embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

One embodiment of the invention provides a probe compound for protein tyrosine phosphatases (PTPs), as shown in Formula (I):

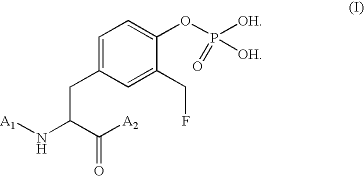

In Formula (I), A₁ and A₂ may represent amino acids.

The amino acids may comprise leucine, phenylalanine, glutamic acid, lysine, alanine, arginine, aspartic acid, asparagine, citrulline, cysteine, cystine, glutamine, glycine, histidine, hydroxyproline, isoleucine, methionine, proline, serine, threonine, tryptophan, valine or a combination thereof.

The probe compound may further comprise a linker, for example 2,2′-(ethylenedioxy)bis(ethylamine), connected to A₁ or A₂.

The probe compound may further comprise a reporter group, for example biotin, connected to the linker.

In the design of the disclosed probe compound, the fluoro-modified tyrosine phosphate is a core structure of a recognition unit. It comprises an ortho-fluoromethyl phosphotyrosine derivative. After hydrolysis, the recognition unit is converted to a highly reactive quinone methide intermediate through 1,4-elimination, which is capable of undergoing alkylation with suitable nucleophilic groups on protein tyrosine phosphatase (PTP). The enzyme specificity of the probe compound is improved by tuning the amino acid sequences (type and length) of the recognition unit. In addition to the fluorine atom, other halogen atoms, for example chlorine, bromine or iodine, are inappropriate for use due to direct reaction with the nucleophilic group of the enzyme, which would form a non-specific alkylation.

One embodiment of the invention provides a probe compound precursor for protein tyrosine phosphatases (PTPs), as shown in Formula (II):

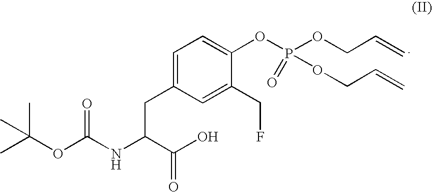

One embodiment of the invention provides a probe compound precursor for protein tyrosine phosphatases (PTPs), as shown in Formula (III):

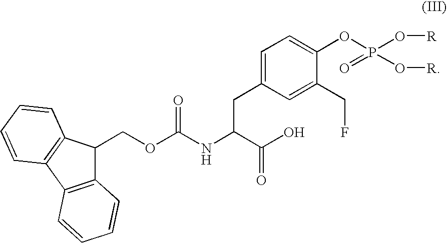

In Formula (III), R may represent —CH₂CH═CH₂ or —CH₂C₆H₅.

The disclosed probe compound precursor of Formula (III) is suitable to be applied in Fmoc chemistry peptide synthesis, for example Fmoc chemistry solid phase peptide synthesis (SPPS). When R=-Bn, the peptide product is cut from a solid phase carrier by treatment with a trifluoroacetic acid (TFA) reagent, which simultaneously removes all the side chain protecting groups of the peptide, simplifying the synthesis processing steps.

One embodiment of the invention provides a probe compound for protein tyrosine phosphatases (PTPs), as shown in Formula (IV):

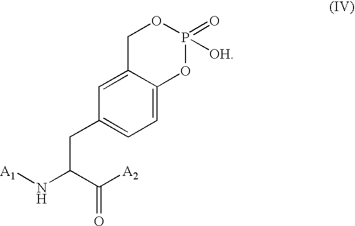

In Formula (IV), A₁ and A₂ may represent amino acids.

The amino acids may comprise leucine, phenylalanine, glutamic acid, lysine, alanine, arginine, aspartic acid, asparagine, citrulline, cysteine, cystine, glutamine, glycine, histidine, hydroxyproline, isoleucine, methionine, proline, serine, threonine, tryptophan, valine or a combination thereof.

The probe compound may further comprise a linker, for example 2,2′-(ethylenedioxy)bis(ethylamine), connected to A₁ or A₂.

The probe compound may further comprise a reporter group, for example biotin, connected to the linker.

One embodiment of the invention provides a probe compound precursor for protein tyrosine phosphatases (PTPs), as shown in Formula (V):

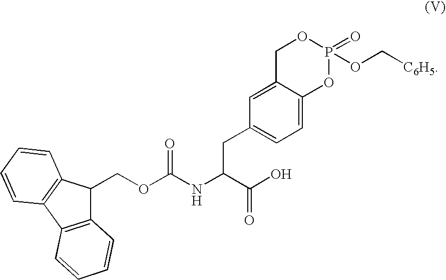

### Example 1

Synthesis of Probe Compound 30a of the Invention

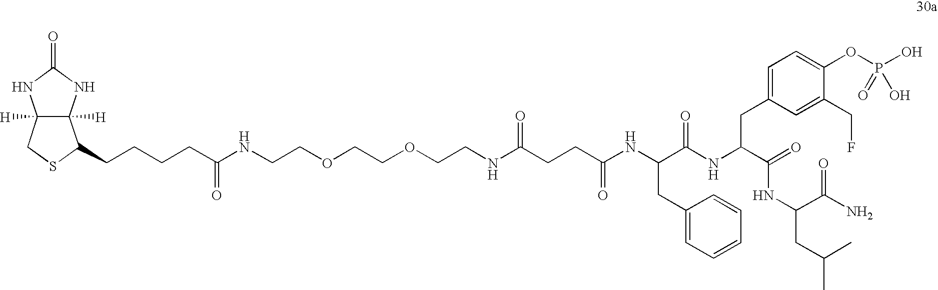

Synthesis Schemes:

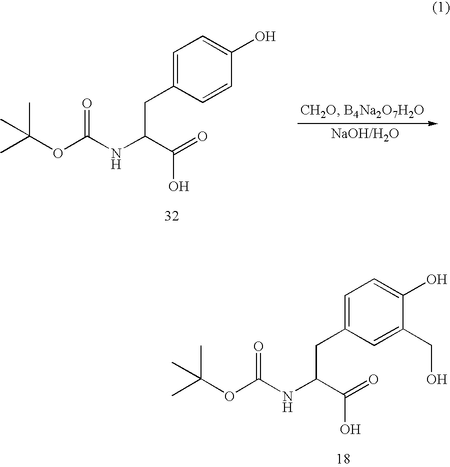

To a solution of Boc-L-Tyr (compound 32) (13.0 g, 46.2 mmol), 1N NaOH (110 mL, 110 mmol), sodium borate decahydrate (44.1 g, 116 mmol) in 180 mL of water was added 35% formaldehyde (19.8 mL, 250 mmol). The reaction mixture was stirred at 40° C. for 3 days. When no more starting material was detected by TLC, the pH was adjusted to three with 1N HCl. The solution was then extracted with EtOAc. The combined organic layer was washed with brine (×2), dried over anhydrous Na₂SO₄, filtered, and concentrated. Compound 18 was obtained (12.2 g, 85%) as oil.

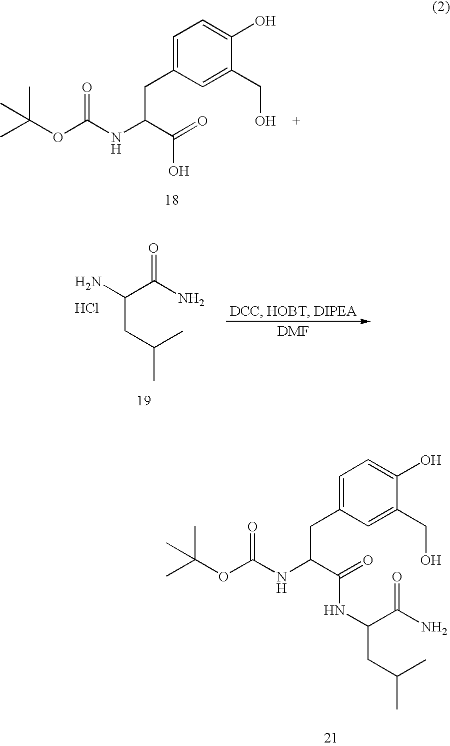

To an ice-cooled solution of compound 18 (704 mg, 2.26 mmol), HOBt (61 mg, 0.45 mmol), L-leucinamide hydrochloride (compound 19) (374 mg, 2.26 mmol), and DIEA (1.495 mL, 9.05 mmol) in 20 mL of anhydrous DMF was added a solution of DCC (513 mg, 2.49 mmol) in 1 mL of DMF. The mixture was allowed to warm to room temperature and stirred for another 16 hr. The white DCU precipitate was filtered off. The filtrate was concentrated to dryness and the residual oil was dissolved in EtOAc and then washed consecutively with 5% citric acid (×1), 5% NaHCO₃ (×3), H₂O (×3), and brine (×2). The organic layer was dried over anhydrous Na₂SO₄, filtered, and concentrated. Compound 21 was obtained (760 mg, 79%) as a white solid after silica gel column chromatography eluted with CHCl₃/MeOH (94/6).

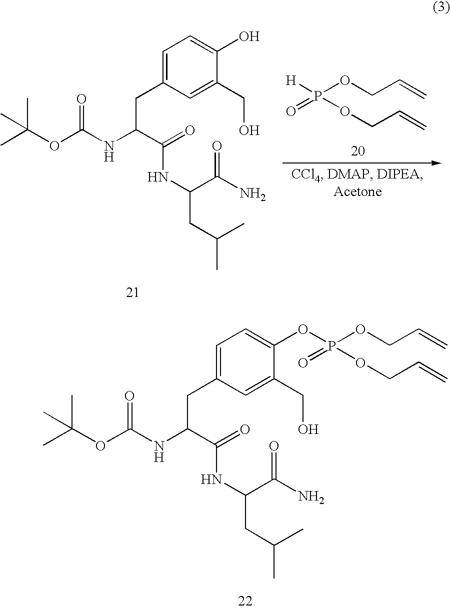

To an ice-cooled solution of compound 21 (2.00 g, 4.72 mmol), DIEA (3.1 mL, 19 mmol), CCl₄ (4.5 mL, 47 mmol), and DMAP (115 mg, 0.943 mmol) in 50 mL of anhydrous acetone was added dropwise diallyl phosphite (compound 20) (1.4 mL, 9.4 mmol). The mixture was allowed to warm to room temperature. After stirring for 18 hr the reaction mixture was concentrated under reduced pressure and the residual oil was dissolved in EtOAc and then washed consecutively with 5% citric acid (×3), H₂O (×3), and brine (×2). The organic layer was dried over anhydrous Na₂SO₄, filtered, and concentrated. Compound 22 was obtained (2.34 g, 85%) as colorless oil after silica gel column chromatography eluted with CHCl₃/MeOH (9/1).

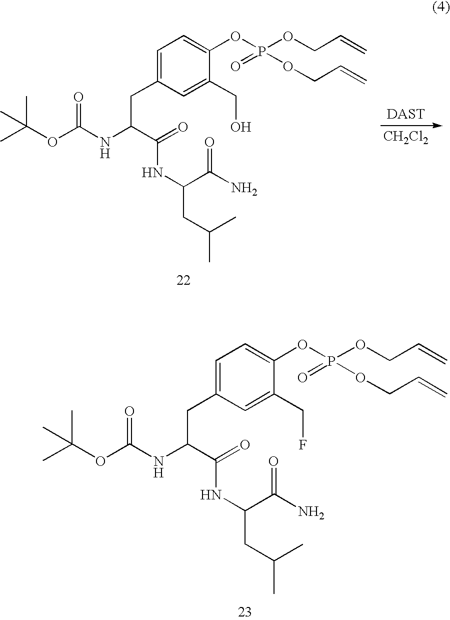

To an ice-cooled solution of compound 22 (1.470 g, 2.52 mmol) in 25 mL of anhydrous CH₂Cl₂ was slowly added DAST (463 mL, 3.78 mmol) through a syringe. The reaction mixture was allowed to warm to room temperature. When no more starting material was observed, it was cooled and quenched by adding 0.5 mL of MeOH and a small amount of silica gel. Silica gel was filtered off and the filtrate was concentrated under reduced pressure. The residual oil was dissolved in EtOAc and then washed consecutively with 5% NaHCO₃ (×3) and brine (×2). The organic layer was dried over anhydrous Na₂SO₄, filtered, and concentrated. The desired product was purified by silica gel column chromatography eluted with CHCl₃/MeOH (95/5) to give compound 23 (738 mg, 50%) as colorless oil.

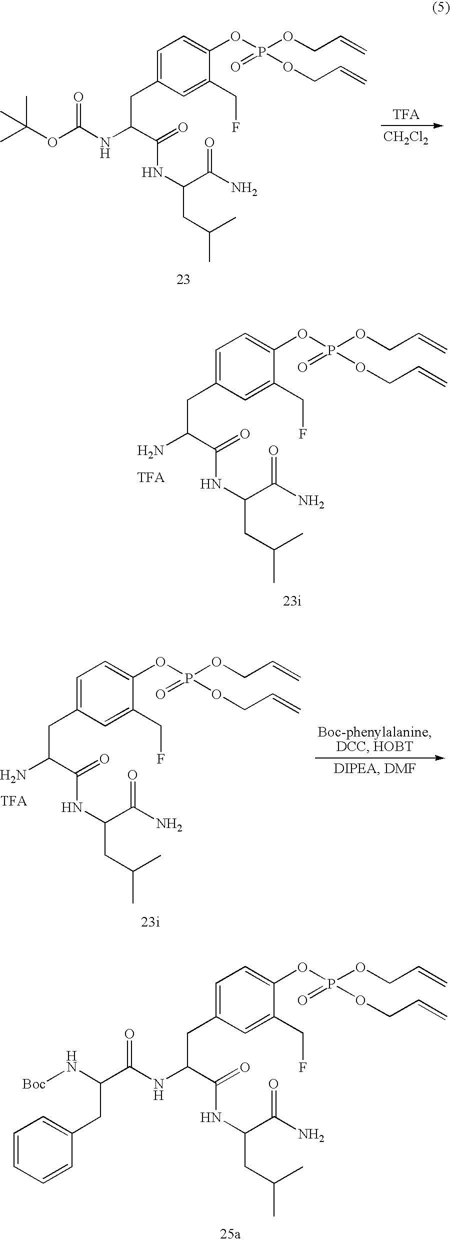

To a solution of the fluorinated compound 23 (1.00 g, 1.71 mmol) in 17 mL CH₂Cl₂ was added 3.4 mL of TFA. After stirring at room temperature for 30 min, the reaction mixture was concentrated under reduced pressure and then kept under high vacuum to remove the residual TFA. The resultant TFA salt (compound 23i) was used for the coupling reaction without further purification. To an ice-cooled solution of the TFA salt (compound 23i), DIEA (1.2 mL, 6.8 mmol), HOBt (93 mg, 0.68 mmol), and Boc-L-Phe (454 mg, 1.71 mmol) in 15 mL of anhydrous DMF was added a solution of DCC (423 mg, 2.05 mmol) in 3 mL of DMF. The mixture was allowed to warm to room temperature and stirred for another 18 hr. The white DCU precipitate was filtered off. The filtrate was concentrated to dryness. The residual oil was dissolved in CHCl₃ and then washed consecutively with 5% citric acid (×3), H₂O (×3), and brine (×2). The organic layer was dried over anhydrous Na₂SO₄, filtered, and concentrated. Compound 25a was obtained (1.102 g, 80%) as a white solid after silica gel column chromatography eluted with CHCl₃/MeOH (92/8).

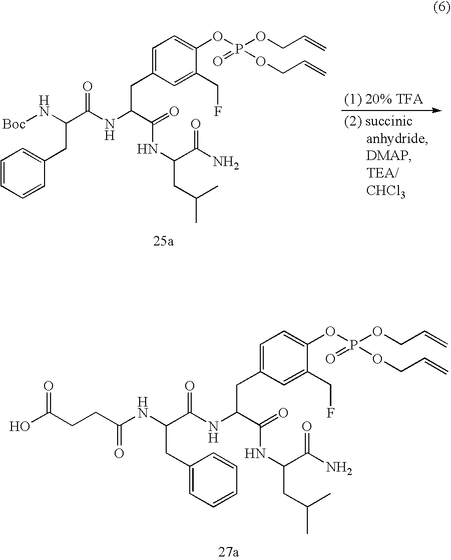

To a solution of compound 25a (607 mg, 0.828 mmol) in 5 mL of CH₂Cl₂ was added 1 mL of TFA. After stirring at room temperature for 30 min, the solvent and acid were removed under reduced pressure to give the TFA salt, which was used for the coupling reaction without further purification. To a solution of the TFA salt in 8 mL of anhydrous CH₂Cl₂ was added TEA (465 mL, 3.35 mmol), DMAP (20 mg, 0.16 mmol), and succinic anhydride (166 mg, 1.66 mmol). After stirring at room temperature for 18 hr, CHCl₃ (50 ml) was added to dilute the reaction mixture. The diluted reaction mixture was then washed consecutively with 5% citric acid (×2), H₂O (×3), and brine (×2). The organic layer was dried over anhydrous Na₂SO₄, filtered, and concentrated. Compound 27a was obtained (426 mg, 70%) as an oil after silica gel column chromatography eluted with CHCl₃/MeOH (9/1).

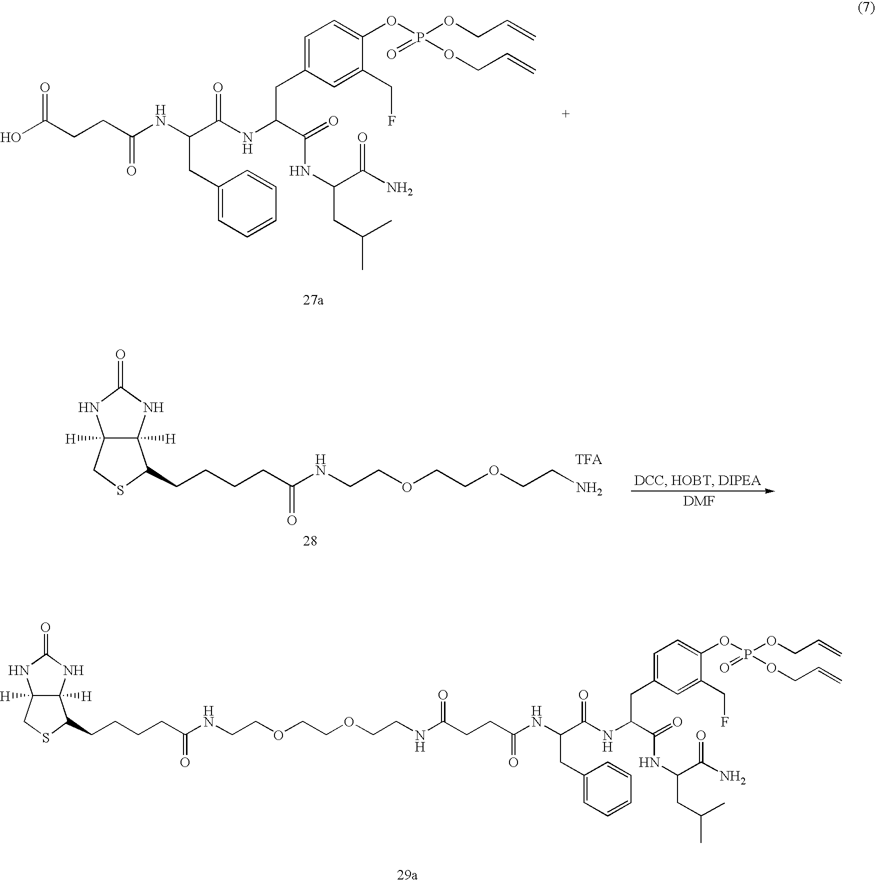

To an ice-cooled solution of compound 28 (386 mg, 0.819 mmol), compound 27a (600 mg, 0.819 mmol), DIEA (1.082 mL, 6.55 mmol) and HOBt (55 mg, 0.41 mmol) in 7 mL of DMF was added a solution of DCC (186 mg, 0.901 mmol) in 1 mL of DMF. The mixture was allowed to warm to room temperature and stirred for another 18 hr. The white DCU precipitate was filtered off. The filtrate was concentrated to dryness. The residual oil was dissolved in CHCl₃ and then washed consecutively with 5% citric acid (×1) and brine (×1). The organic layer was dried over anhydrous Na₂SO₄, filtered, and concentrated. Compound 29a was obtained (580 mg, 65%) as oil after silica gel column chromatography eluted with CHCl₃/MeOH (9/1).

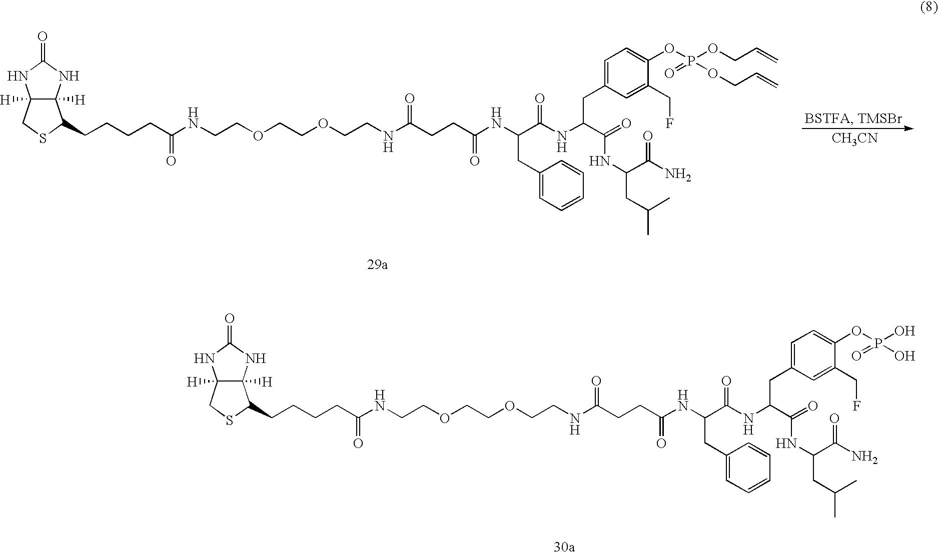

To an ice-cooled solution of compound 29a (20.0 mg, 0.0184 mmol) and BSTFA (99 mL, 0.37 mmol) in 1 mL of anhydrous CH₃CN was slowly added TMSBr (24 mL, 0.183 mmol). The reaction mixture was allowed to warm to room temperature and stirred further for 45 min. The reaction was quenched with 50% TEA in MeOH (1 mL). The organic solvents were removed under reduced pressure to give the crude product, which was purified by chromatography over Sephadex LH-20 eluted with MeOH. The fractions containing the product were pooled, concentrated and then lyophilized to afford compound 30a (18 mg, 90%) as a colorless powder.

¹H-NMR: (CD₃OD, 400 MHz) δ 7.46-7.39 (m, 2H, aromatic), 7.33-7.19 (m, 6H, aromatic), 5.58 (d, J=47.6 Hz, 2H, CH₂F), 4.52 (m, 2H), 4.45 (m, 1H), 4.39-4.31 (m, 2H), 3.64 (s, 4H), 3.60-3.55 (m, 4H), 3.47 (m, 1H), 3.42-3.35 (m, 3H), 3.23-3.18 (m, 8H), 3.09 (dd, J=14.2, 4.4 Hz, 1H), 2.95 (dd, J=12.7, 4.9 Hz, 1H), 2.82 (dd, J=14.1, 10.0 Hz, 1H), 2.75-2.65 (m, 2H), 2.57-2.48 (m, 2H), 2.34 (m, 1H), 2.25 (m, 2H), 1.81-1.57 (m, 8H), 1.47 (m, 2H), 1.33 (t, J=7.3 Hz, 9H, TEA), 1.00 (d, J=5.4 Hz, 3H), 0.93 (d, J=5.4 Hz, 3H); ¹³C-NMR: (CD₃OD, 100 MHz) δ 177.5 (C), 176.4 (C), 176.2 (C), 174.7 (C), 174.6 (C), 173.7 (C), 166.1 (C), 151.0 (C), 138.4 (C), 133.5 (C), 131.1 (CH), 130.1 (CH), 130.0 (CH), 129.7 (C), 129.5 (CH), 127.8 (CH), 121.8 (CH), 81.3 (d, J=162.3 Hz, CH₂F), 71.3 (CH₂), 71.2 (CH₂), 70.6 (CH₂), 63.4 (CH), 61.6 (CH), 57.5 (CH), 57.0 (CH), 53.1 (CH), 53.0 (CH), 47.7 (CH₂, TEA), 41.5 (CH₂), 41.1 (CH₂), 40.5 (CH₂), 40.2 (CH₂), 37.9 (CH₂), 36.9 (CH₂), 36.7 (CH₂), 31.9 (CH₂), 31.7 (CH₂), 29.8 (CH₂), 29.5 (CH₂), 26.8 (CH₂), 25.7 (CH), 23.7 (CH₃), 21.5 (CH₃), 9.2 (CH₃, TEA); ³¹P-NMR: (D₂O, 400 MHz) δ −3.89; ¹⁹F-NMR: (CD₃OD, 400 MHz) δ −217.5 (t, J=50.0 Hz); IR (neat): 3377, 3291, 2913, 2853, 1633, 1547, 1467, 1255, 1090; HRMS calcd for C₄₅H₆₆FN₈O₁₃PSNa: 1031.4089, found: 1031.4070.

### Example 2

Synthesis of Probe Compound 30b of the Invention

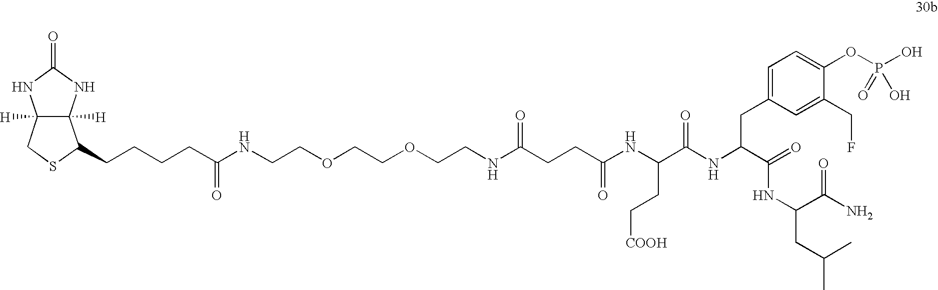

Synthesis Schemes:

Schemes (1)-(4) are similar to Example 1.

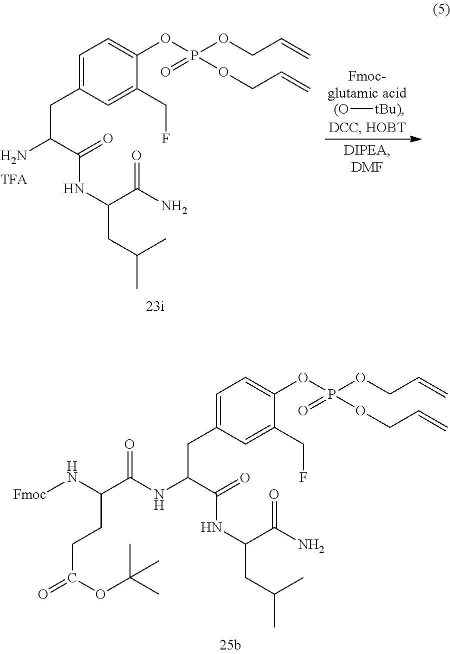

The same procedure as that for compound 25a was used, except Fmoc-Glu(OtBu)-OH was used for the coupling. Yield 75%.

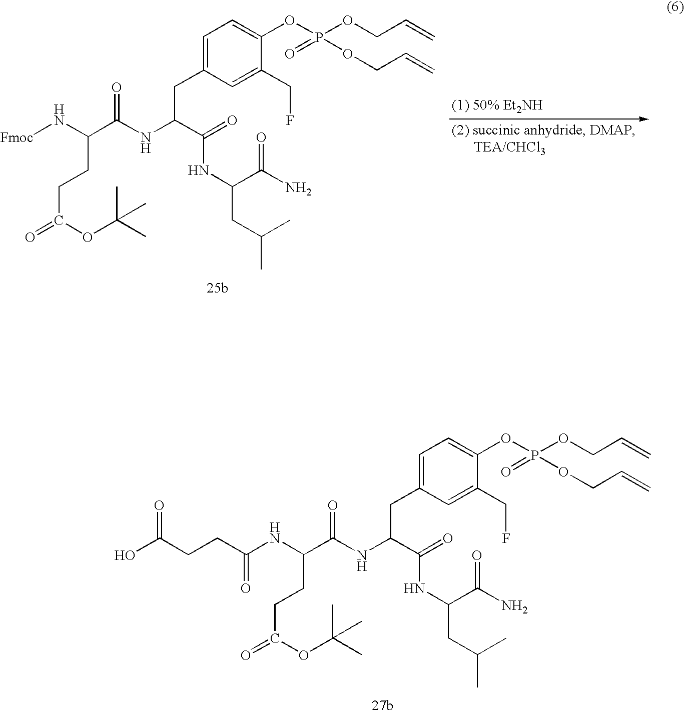

To a solution of compound 25b (203 mg, 0.227 mmol) in 4 mL of CH₂Cl₂ was added 2 mL of Et₂NH. The mixture was stirred at room temperature for 30 min. When no more starting material was observed by TLC analysis, the reaction mixture was concentrated under reduced pressure and then kept under high vacuum to remove the residual Et₂NH. To a solution of the resultant Et₂NH salt in 2 mL of anhydrous CH₂Cl₂ was added succinic anhydride (46 mg, 0.46 mmol) at room temperature. After stirring for 18 hr, CHCl₃ (20 ml) was added to dilute the reaction mixture. The diluted reaction mixture was then washed consecutively with 5% citric acid (×2), H₂O (×3), and brine (×2). The organic layer was dried over anhydrous Na₂SO₄, filtered, and concentrated. Compound 27b was obtained (105 mg, 60%) as oil after silica gel column chromatography eluted with CHCl₃/MeOH (9/1).

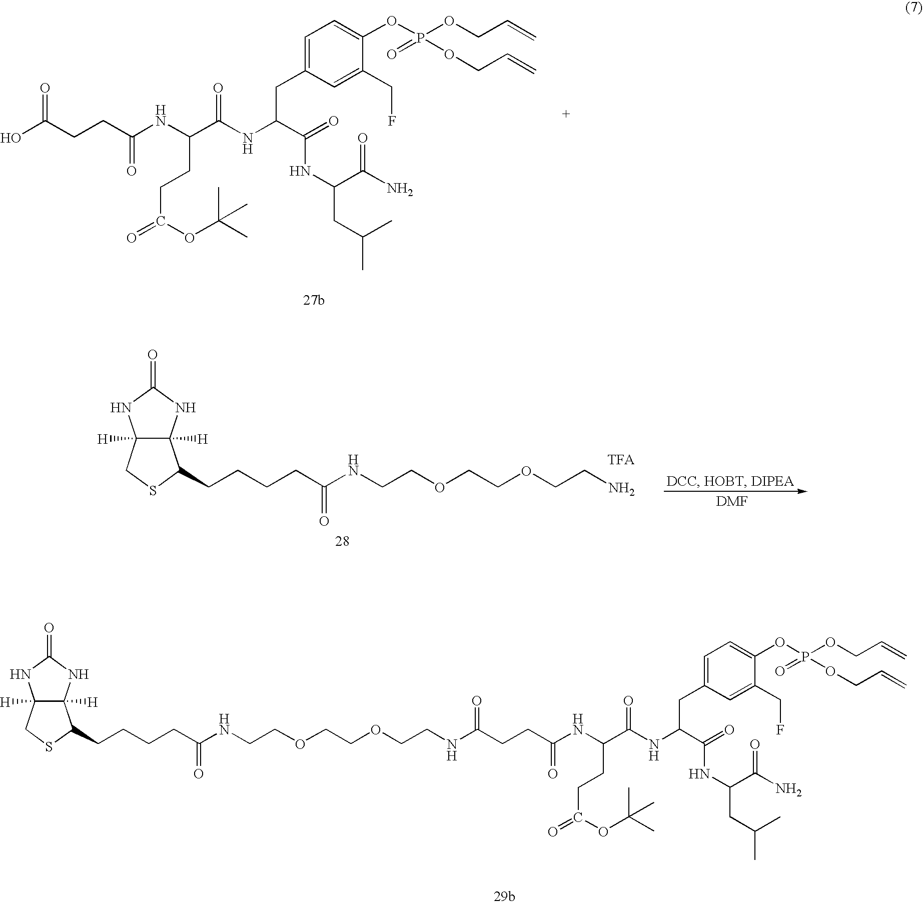

The same procedure as that for compound 29a was used. Yield 60%.

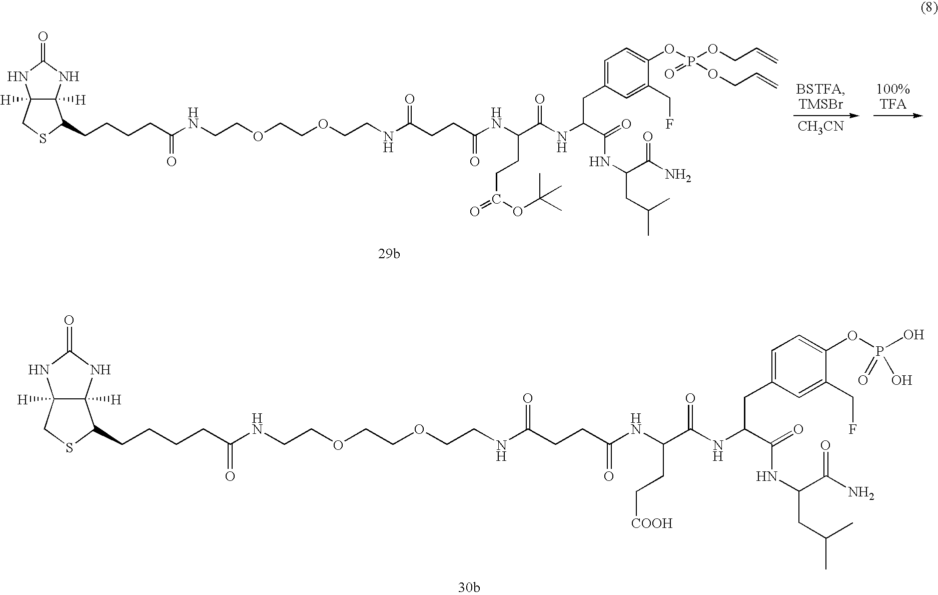

The same procedure as that for compound 30a was used. Yield 80%.

¹H-NMR: (CD₃OD, 400 MHz) δ 7.45-7.40 (m, 2H, aromatic), 7.31 (d, J=7.4 Hz, 1H, aromatic), 5.57 (d, J=47.5 Hz, 2H, CH₂F), 4.54-4.49 (m, 2H), 4.40-4.32 (m, 2H), 4.16 (m, 1H), 3.64 (s, 4H), 3.61-3.56 (m, 4H), 3.47 (m, 1H), 3.43-3.36 (m, 3H), 3.28-3.16 (m, 12H), 2.96 (dd, J=12.7, 5.0 Hz, 1H), 2.79-2.72 (m, 2H), 2.61-2.54 (m, 2H), 2.44 (m, 1H), 2.32-2.12 (m, 4H), 2.01 (m, 1H), 1.87 (m, 1H), 1.81-1.58 (m, 8H), 1.51-1.43 (m, 2H), 1.34 (t, J=7.3 Hz, 15H, TEA), 1.00 (d, J=5.9 Hz, 3H), 0.93 (d, J=5.9 Hz, 3H); ¹³C-NMR: (CD₃OD, 100 MHz) δ 178.2 (C), 177.6 (C), 176.6 (C), 176.2 (C), 175.1 (C), 174.6 (C), 173.9 (C), 166.1 (C), 151.0 (C), 133.7 (C), 131.0 (CH), 130.0 (CH), 129.6 (C), 121.7 (CH), 81.3 (d, J=162.6 Hz, CH₂F), 71.4 (CH₂), 71.2 (CH₂), 70.5 (CH₂), 63.3 (CH), 61.6 (CH), 57.6 (CH), 57.4 (CH), 56.0 (CH), 53.1 (CH), 47.5 (CH₂, TEA), 41.5 (CH₂), 41.1 (CH₂), 40.5 (CH₂), 40.2 (CH₂), 36.7 (CH₂), 36.5 (CH₂), 32.3 (CH₂), 31.8 (CH₂), 29.8 (CH₂), 29.5 (CH₂), 27.5 (CH₂), 26.8 (CH₂), 25.7 (CH), 23.8 (CH₃), 21.4 (CH₃), 9.1 (CH₃); ³¹P-NMR: (D₂O, 400 MHz) δ −3.96; ¹⁹F-NMR: (CD₃OD, 400 MHz) δ −214.6 (t, J=50.0 Hz); IR (neat): 3277, 2919, 2846, 2661, 1733, 1633, 1547, 1414, 1262, 1149, 1030; HRMS calcd for C₄₁H₆₄FN₈O₁₅PSNa: 1013.3831, found: 1013.3817.

### Example 3

Synthesis of Probe Compound 30c of the Invention

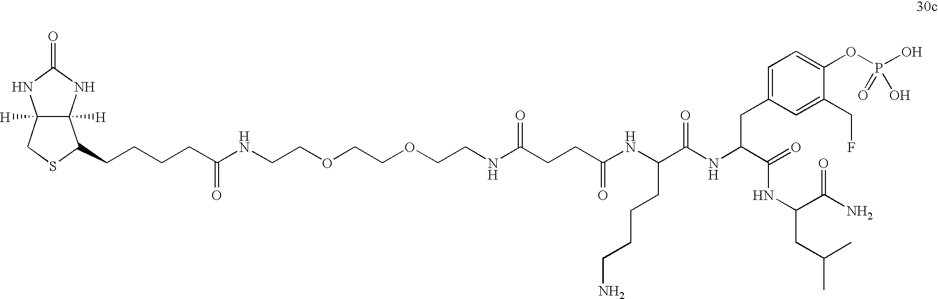

Synthesis Schemes:

Schemes (1)-(4) are similar to Example 1.

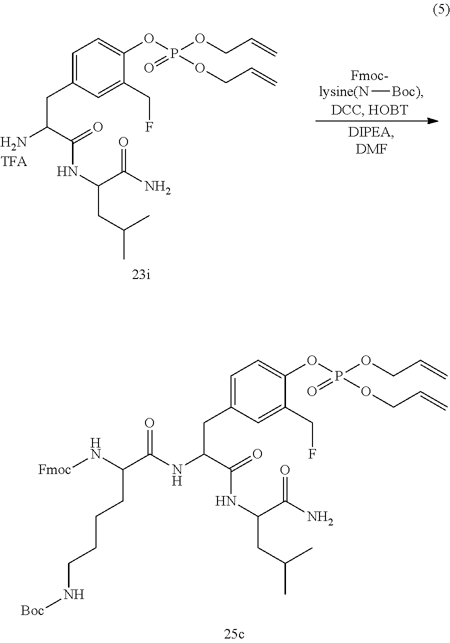

The same procedure as that for compound 25b was used, except Fmoc-Lys(Boc)-OH was used for the coupling. Yield 78%.

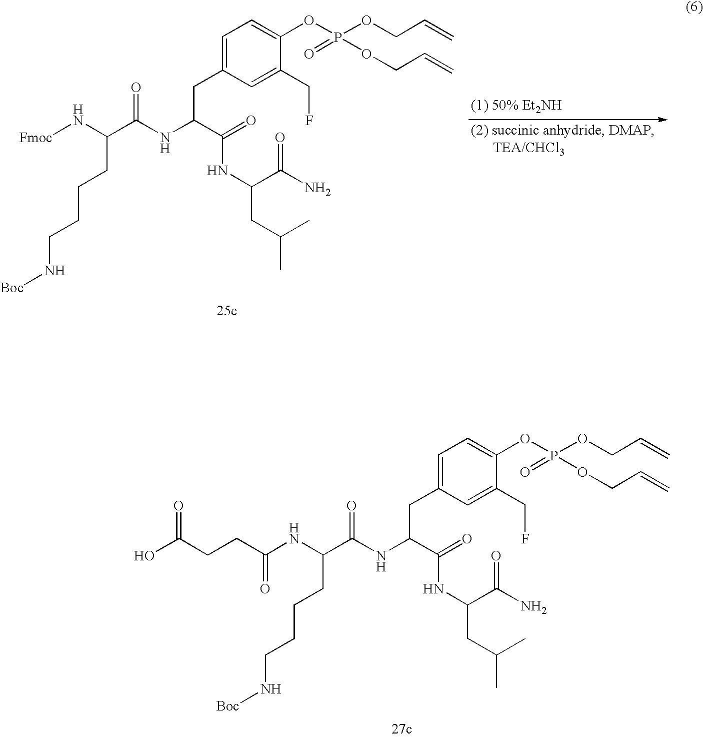

The same procedure as that for compound 27b was used. Yield 65%.

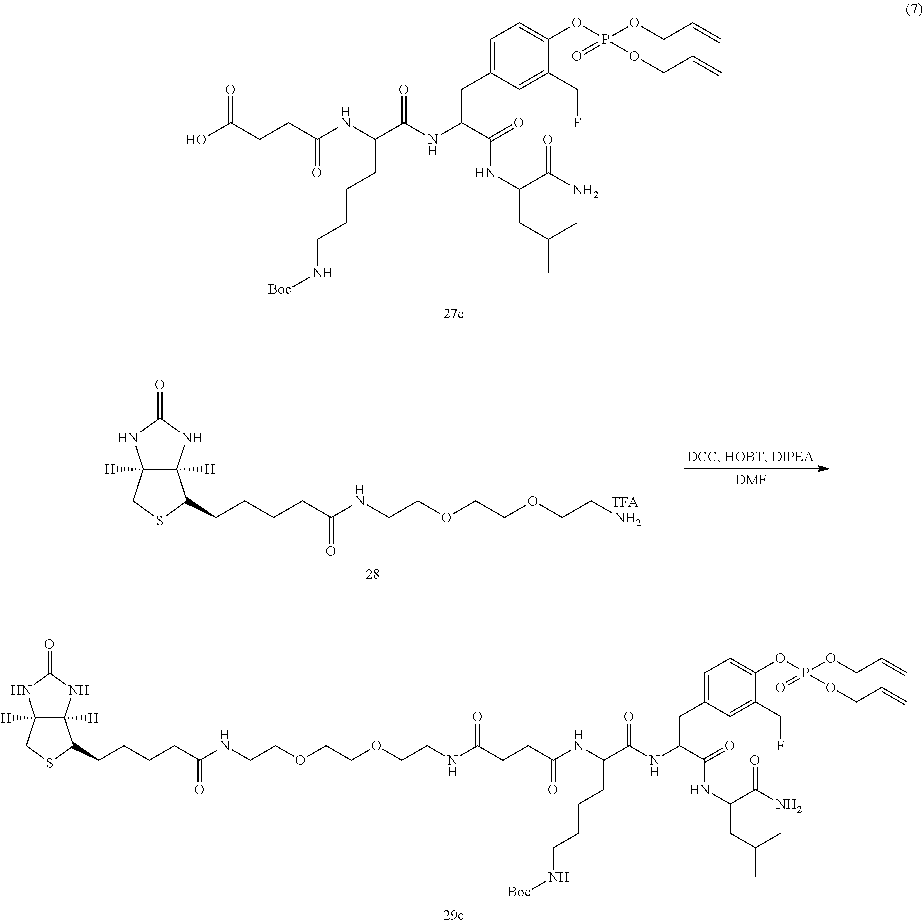

The same procedure as that for compound 29a was used. Yield 60%.

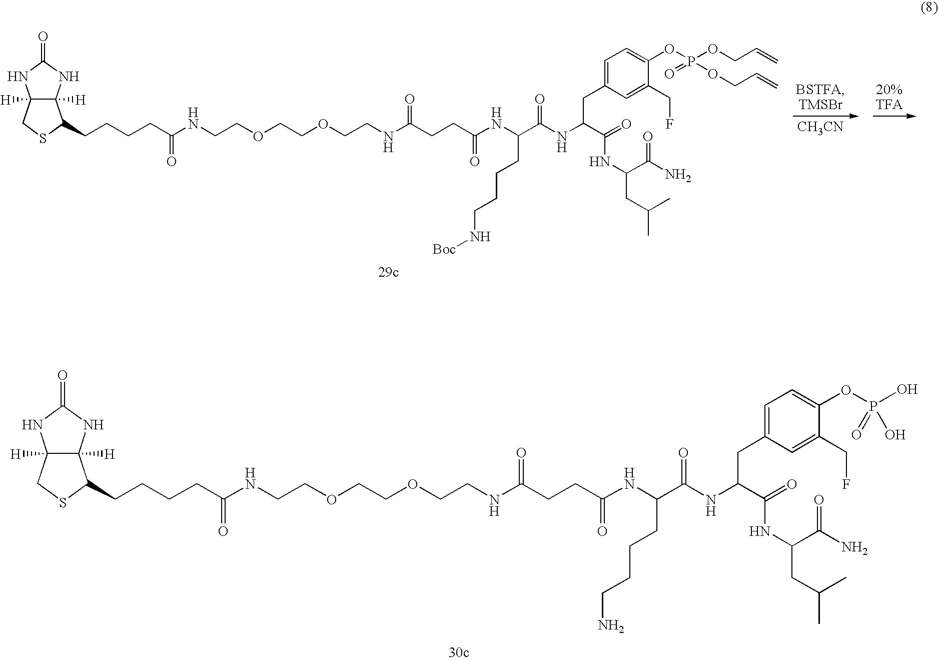

The same procedure as that for compound 30a was used. Yield 80%.

¹H-NMR: (D₂O, 400 MHz) δ 7.38 (s, 1H, aromatic), 7.34-7.29 (m, 2H, aromatic), 5.50 (d, J=47.5 Hz, 2H, CH₂F), 4.64-4.54 (m, 2H), 4.36 (m, 1H), 4.27 (m, 1H), 4.09 (m, 1H), 3.64 (s, 4H), 3.63-3.56 (m, 4H), 3.47-3.31 (m, 4H), 3.28-3.20 (m, 2H), 3.17 (q, J=7.3 Hz, 2H, TEA), 3.03 (m, 1H), 2.94 (m, 1H), 2.79 (m, 2H), 2.72 (m, 1H), 2.62-2.46 (m, 3H), 2.22 (m, 2H), 1.70-1.48 (m, 12H), 1.40-1.32 (m, 2H), 1.25 (t, J=7.3 Hz, 3H, TEA), 1.10 (m, 1H), 0.96 (m, 1H), 0.92 (d, J=5.7 Hz, 3H), 0.84 (d, J=5.6 Hz, 3H); ¹³C-NMR: (CD₃OD, 100 MHz) δ 177.7 (C), 177.2 (C), 176.1 (C), 175.2 (C), 174.6 (C), 174.0 (C), 166.1 (C), 151.3 (C), 133.8 (C), 131.3 (CH), 130.6 (CH), 129.0 (C), 121.3 (CH), 81.4 (d, J=163.5 Hz, CH₂F), 71.4 (CH₂), 71.2 (CH₂), 70.6 (CH₂), 70.5 (CH₂), 63.4 (CH), 61.6 (CH), 57.8 (CH), 57.0 (CH), 56.6 (CH), 53.2 (CH), 47.6 (CH₂, TEA), 41.5 (CH₂), 41.1 (CH₂), 40.6 (CH₂), 40.2 (CH₂), 36.7 (CH₂), 36.3 (CH₂), 32.1 (CH₂), 31.8 (CH₂), 30.7 (CH₂), 29.8 (CH₂), 29.5 (CH₂), 28.0 (CH₂), 26.9 (CH₂), 25.8 (CH), 23.8 (CH₃), 22.5 (CH₂), 21.4 (CH₃), 9.2 (CH₃, TEA); ³¹P-NMR: (D₂O, 400 MHz) δ −3.82; ¹⁹F-NMR: (CD₃OD, 400 MHz) δ −214.5 (t, J=50.0 Hz); IR (neat): 3284, 2919, 2860, 1686, 1633, 1554, 1467, 1255, 1103; HRMS calcd for C₄₂H₆₉FN₉O₁₃PS: 990.4535, found: 990.4563.

### Example 4

Synthesis of Precursor Compound 47 of the Invention

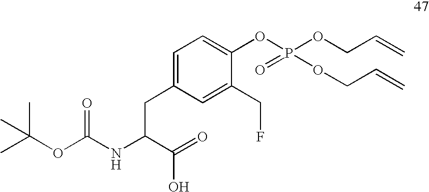

Synthesis Schemes:

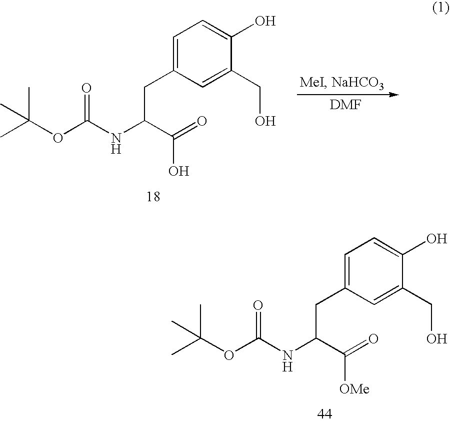

230.6 mg (0.7414 mmol) of compound 18 was dissolved in 6 ml of DMF in a flask. After adding 124.6 mg (1.483 mmol) of NaHCO₃, 0.93 ml (1.48 mmol) of MeI was slowly added under room temperature and reacted for 12 hours. After removal of DMF, the resulting solution was mixed with ethyl acetate and extracted with 5% citric acid aqueous solution for three times. Next, the organic layer was extracted with saturated NaCl aqueous solution twice, dried with dried Na₂SO₄, concentrated and separated with a silica gel chromatography column (hexane:EtOAc=3:7) to form 144.7 mg of oily compound 44 with a yield of 60%.

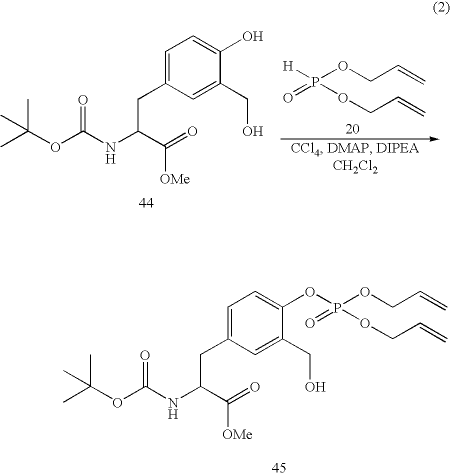

214 mg (0.659 mmol) of compound 44 and 6 ml of dichloromethane were added in a dried 25 ml round-bottom flask. 637 μl (6.59 mmol) of tetrachloromethane, 432.2 μl (2.634 mmol) of DIPEA and 16 mg (0.13 mmol) of DMAP were then added under an ice bath for 15 minutes. Next, 194 μl (1.32 mmol) of compound 20 (diallyl phosphite) was slowly added to the flask under room temperature and reacted for 18 hours. After removal of dichloromethane, the resulting solution was mixed with 50 ml of ethyl acetate and respectively extracted with 5% citric acid aqueous solution and distilled water for three times. Next, the organic layer was extracted with saturated NaCl aqueous solution twice, dried with dried Na₂SO₄, concentrated and separated with a silica gel chromatography column (CHCl₃:MeOH=92:8) to form 223.7 g of oily compound 45 with a yield of 70%.

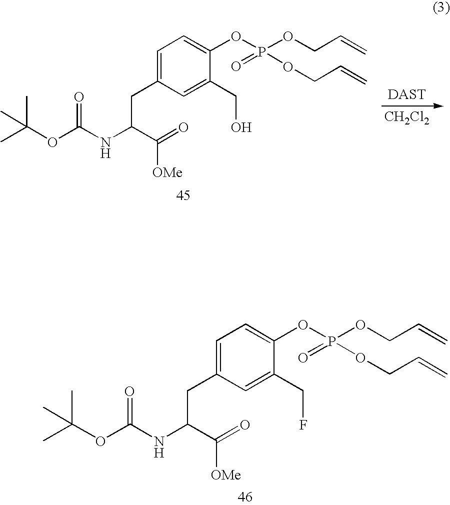

153 mg (0.315 mmol) of compound 45 and 4 ml of dichloromethane were added in a dried 10 ml round-bottom flask and stirred under an ice bath for 15 minutes. 58 μl (0.47 mmol) of DAST was then slowly added along the flask wall and reacted for 6 hours. Next, a small quantity of silica gel was added and stirred for 15 minutes. 0.5 ml of methanol was then added and stirred for 10 minutes. After being filtered and concentrated, the filtrate was mixed with ethyl acetate and extracted with 5% NaHCO₃ aqueous solution for three times. Next, the organic layer was extracted with saturated NaCl aqueous solution twice, dried with dried Na₂SO₄, concentrated and separated with a silica gel chromatography column (CHCl₃:MeOH=9:1) to form 67 mg of oily compound 46 with a yield of 43%.

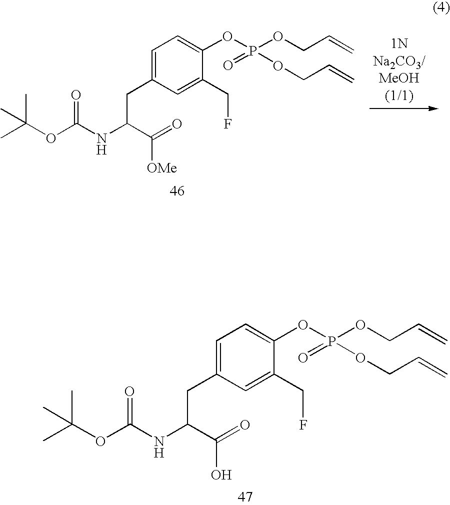

767 mg (1.57 mmol) of compound 46 and 9.4 ml of methanol were added in a dried 50 ml round-bottom flask and stirred for 5 minutes. 9.4 ml of 1N Na₂CO₃ was then added and reacted for 1 hour. After adding 20 ml of ethyl acetate, 5% citric acid aqueous solution was slowly added to adjust pH to 2-3. Next, the organic layer was extracted with saturated NaCl aqueous solution twice, dried with dried Na₂SO₄, concentrated and separated with a silica gel chromatography column (CHCl₃:MeOH=85:15) to form 596 mg of oily compound 47 with a yield of 80%.

¹H-NMR: (acetone-d₆, 400 MHz) δ 7.42 (s, 1H, aromatic), 7.40-7.30 (m, 2H, aromatic), 6.15 (d, J=8.4 Hz, 1H, NH), 5.99 (m, 2H), 5.50 (d, J=47.6 Hz, 2H, CH₂F), 5.39 (dd, J=17.2, 1.4 Hz, 2H), 5.25 (dd, J=10.5, 1.4 Hz, 2H), 4.70-4.66 (m, 4H), 4.43 (m, 1H), 3.23 (dd, J=13.9, 4.7 Hz, 1H), 3.03 (dd, J=13.9, 9.1 Hz, 1H), 1.36 (s, 9H); ¹³C-NMR: (CDCl₃, 100 MHz) δ 173.7 (C), 155.2 (C), 147.0 (d, J=4.7 Hz, C), 133.7 (C), 131.6 (CH), 131.0 (CH), 130.5 (CH), 127.3 (d, J=17.2 Hz, C), 119.7 (CH), 118.9 (CH₂), 80.0 (C), 79.6 (d, J=165.8 Hz, CH₂F), 69.1 (CH₂), 54.0 (CH), 40.0 (CH₂), 28.1 (CH₃); ³¹P-NMR: (CDCl₃, 400 MHz) δ −6.18; ¹⁹F-NMR: (acetone-d₆, 400 MHz) δ −214.6 (t, J=50.0 Hz); IR (neat): 3430, 3330, 2979, 2919, 1719, 1501, 1375, 1255, 1215, 1169, 1036, 970; HRMS calcd for C₂₁H₂₉FNO₈PNa: 496.1513, found: 496.1515.

### Example 5

Synthesis of Precursor Compound 49 of the Invention

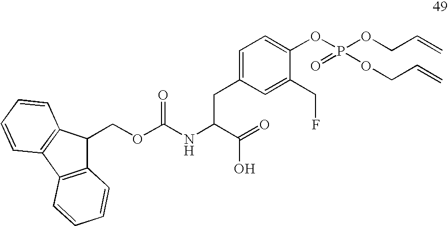

Synthesis Schemes:

Schemes (1)-(4) are similar to Example 4.

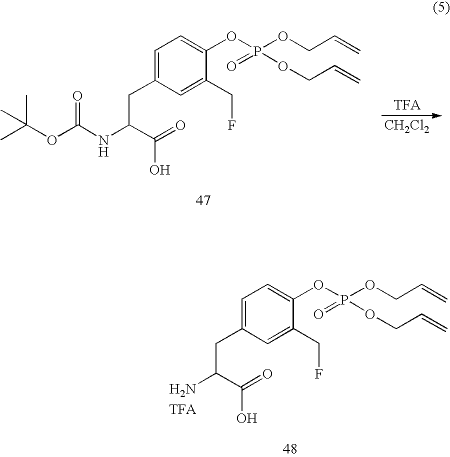

200 mg (0.422 mmol) of compound 47 and 5 ml of dichloromethane were added in a dried 25 ml round-bottom flask. 1 ml of TFA was then added and stirred for 30 minutes. A small quantity of toluene was repeatly added and removed for 3 times. After vacuum drying for 30 minutes, compound 48 was formed.

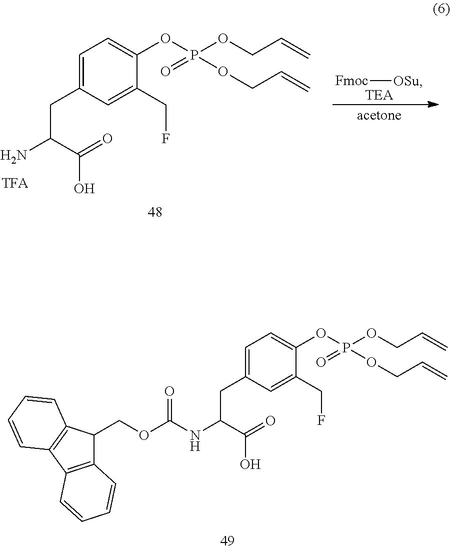

Compound 48 was dissolved in 5 ml of acetone in a flask. 118.8 μl (0.8448 mmol) of TEA and 213.7 mg (0.6336 mmol) of Fmoc-OSu were added and reacted for 18 hours. After filtration and removal of solvent, the filtrate was mixed with 50 ml of trichloromethane and respectively extracted with 5% citric acid aqueous solution and distilled water. Next, the organic layer was extracted with saturated NaCl aqueous solution twice, dried with dried Na₂SO₄, concentrated and separated with a silica gel chromatography column (CHCl₃:MeOH=85:15) to form 146.8 mg of oily compound 49 with a yield of 60%.

¹H-NMR: (CD₃OD, 400 MHz) δ 7.82 (d, J=7.5 Hz, 2H, aromatic), 7.63 (m, 2H, aromatic), 7.44-7.40 (m, 3H, aromatic), 7.34-7.24 (m, 4H, aromatic), 5.96 (m, 2H), 5.43 (d, J=47.6 Hz, 2H, CH₂F), 5.38 (d, J=15.9 Hz, 2H), 5.27 (d, J=10.4 Hz, 2H), 4.64 (m, 4H), 4.41-4.35 (m, 2H), 4.23-4.15 (m, 2H), 3.28 (dd, J=13.6, 4.2 Hz, 1H), 2.99 (dd, J=13.6, 9.1 Hz, 1H); ¹³C-NMR: (CD₃OD, 100 MHz) δ 177.1 (C), 158.2 (C), 148.5 (dd, J=6.7, 4.2 Hz, C), 145.1 (C), 142.4 (C), 136.9 (C), 133.2 (CH), 132.4 (CH), 132.2 (CH), 128.7 (CH), 128.5 (d, J=6.8 Hz, C), 128.1 (CH), 126.2 (CH), 120.9 (CH), 120.7 (CH), 119.2 (CH₂), 80.8 (d, J=164.7 Hz, CH₂F), 70.4 (CH₂), 67.9 (CH₂), 57.5 (CH), 48.2 (CH), 38.1 (CH₂); ³¹P-NMR: (CD₃OD, 400 MHz) δ −6.26; ¹⁹F-NMR: (acetone-d₆, 400 MHz) δ −214.7 (t, J=50.0 Hz); IR (neat): 3291, 2946, 1713, 1501, 1448, 1255, 1209, 1036, 970; HRMS calcd for C₃₁H₃₁FNO₈PNa: 618.1669, found: 618.1658.

### Example 6

Synthesis of Precursor Compound 50 (8) of the Invention

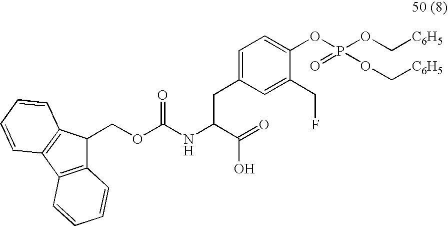

Synthesis Schemes:

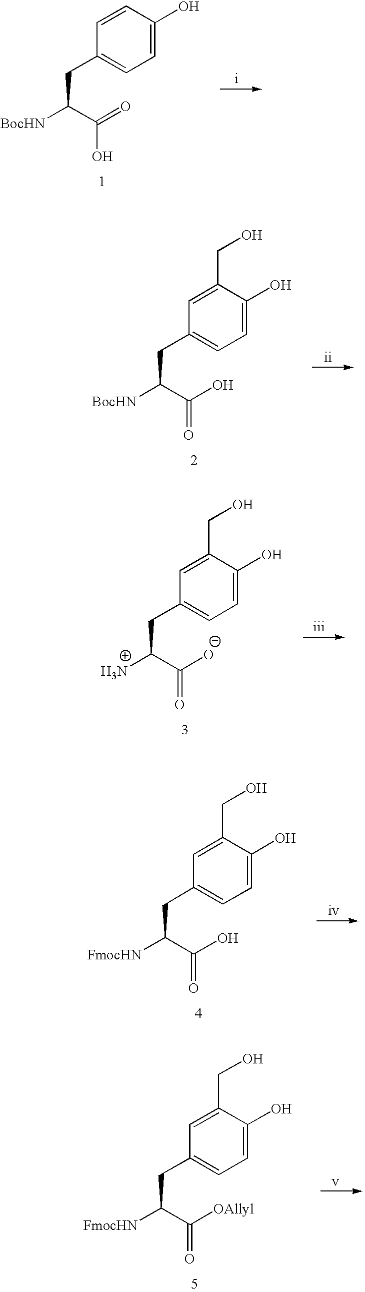

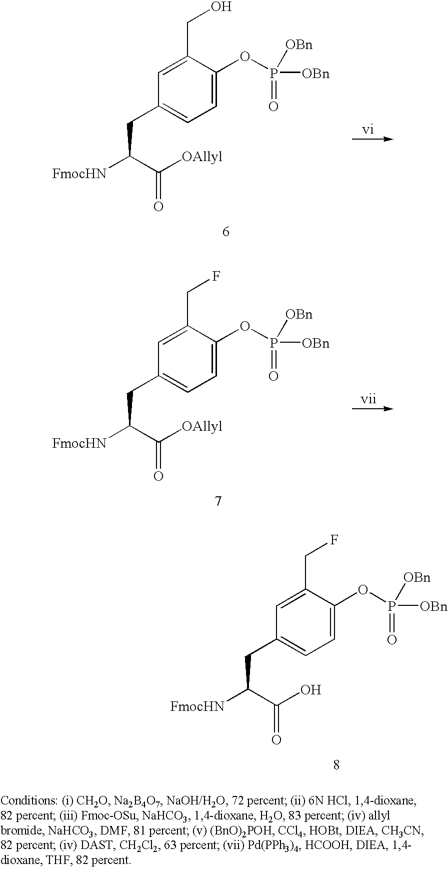

To a solution of Boc-L-Tyr (compound 1) (100 g, 356 mmol), NaOH (28.4 g, 711 mmol), sodium borate decahydrate (298 g, 782 mmol) in 710 mL of water was added 37% formaldehyde (120 mL, 1.600 mmol). The reaction mixture was stirred at 60° C. for 10 hr. When no more starting material was detected by TLC, the pH was adjusted to 3 with 3N HCl. The solution was then extracted with EtOAc. After separation of the organic layer, it was washed with H₂O (×3) and brine (×2), dried over anhydrous Na₂SO₄, filtered, and concentrated. Compound 2 was obtained (79.7 g, 72%) as a white solid after crystallized with EtOAc/ether.

To a solution of compound 2 (5.00 g, 16.1 mmol) in 15 mL of 1,4-dioxone was slowly added 15 mL 6N HCl. After stirring at room temperature for 3 hr no more starting material was detected by TLC, the solution was added 50 ml water and the pH was adjusted to 7 with NaHCO₃. After evaporation of the solvent under reduced pressure, the residue was purified by a reversed phase C18 column chromatography eluted with MeOH/H₂O. The fractions containing the product were pooled, concentrated, and then lyophilized to afford compound 3 as a white solid power (2.79 g, 82%).

9-fluorenylmethyl N-succinimidyl carbonate (4.45 g, 13.2 mmol) and compound 3 (2.92 g, 13.2 mmol) were suspended in 60 mL of 1,4-dioxone and 60 ml water, and the mixture was added NaHCO₃ (3.32 g, 39.6 mmol) at room temperature for 16 hr. The reaction mixture was poured into water (50 mL) and 50 ml 5% NaHCO₃ extracted with EtOAc (×2). The aqueous phase was acidified while being vigorously stirred with concentrated 6N HCl to reach pH 3, and then the aqueous phase was extracted with EtOAc. The organic phase was washed with consecutively with H₂O (×3) and brine (×2). The organic layer was dried over anhydrous Na₂SO₄, filtered, and concentrated. Compound 4 was obtained (4.73 g, 83%) as a white solid after crystallized with EtOAc/CH₂Cl₂.

To a solution of compound 4 (4.50 g, 10.4 mmol) and NaHCO₃ (3.49 g, 41.5 mmol) in 52 mL of DMF was added allyl bromide (2.52 g, 20.8 mmol). After stirring for 48 hr the reaction mixture was dissolved in EtOAc and then washed consecutively with H₂O, 5% citric acid and brine. The organic layer was evaporated to dryness in vacuum and the residue was dissolved in EtOAc and then washed consecutively with H₂O (×3) and brine (×2). The organic layer was dried over anhydrous Na₂SO₄, filtered, and concentrated. Compound 5 was obtained (4.00 g, 81%) as a white solid after crystallized with EtOAc/ether/hexane.

To an ice-cooled solution of compound 5 (1.00 g, 2.11 mmol), CCl₄ (1.0 mL, 10 mmol), HOBt (57 mg, 0.42 mmol) and DIEA (768 L, 4.65 mmol) in 10 mL of anhydrous CH₃CN was added dropwise 95% dibenzyl phosphite (494 L, 2.11 mmol). After stirring for 2 hr the reaction mixture was dissolved in EtOAc and then washed consecutively with 5% citric acid (×2), 5% NaHCO₃ and brine. The organic layer was dried over anhydrous Na₂SO₄, filtered, and concentrated. Compound 6 was obtained (1.27 g, 82%) as colorless oil after silica gel column chromatography eluted with 10-30% gradient EtOAc in CH₂Cl₂.

To an ice-cooled solution of compound 6 (1.23 g, 1.68 mmol) in 11 mL of anhydrous CH₂Cl₂ was slowly added DAST (411 L, 3.36 mmol) through a syringe. The reaction mixture was allowed to warm to room temperature. After 1 hr no more starting material was observed, it was cooled and quenched by adding 0.5 mL of MeOH and a small amount of silica gel. Silica gel was filtered off and the filtrate was concentrated under reduced pressure. The residual oil was dissolved in EtOAc and then washed consecutively with 5% citric acid, 5% NaHCO₃, H₂O and brine. The organic layer was dried over anhydrous Na₂SO₄, filtered, and concentrated. Compound 7 was obtained (780 mg, 63%) as colorless oil after silica gel column chromatography eluted with 30-50% gradient EtOAc in hexane.

To a solution of compound 7 (4.59 g, 6.25 mmol) in 62 mL of 1,4-dioxane/THF (1/1) was sequentially added HCOOH (707 L, 18.7 mmol), DIEA (3.097 L, 18.7 mmol), and Pd(PPh₃)₄ (361 mg, 0.312 mmol). After stirring for 6 hr the reaction mixture was dissolved in EtOAc and then washed consecutively with 5% citric acid, 5% NaHCO₃, H₂O and brine. The organic layer was dried over anhydrous Na₂SO₄, filtered, and concentrated. The residue was first subjected to silica gel column chromatography eluted with CH₂Cl₂/MeOH (95/5), and was purified by reversed phase C18 column chromatography eluted with MeOH/H2O to afford compound 8 (3.55 g, 82%) as a colorless foam.

¹H-NMR (400 MHz, Acetone-d₆): δ 7.84 (d, J=7.5 Hz, 2H, aromatic), 7.65 (d, J=7.6 Hz, 2H, aromatic), 7.48-7.23 (m, 17H, aromatic), 6.79 (d, J=8.6 Hz, 1H, NH), 5.40 (d, J=47.6 Hz, 2H, CH₂F), 5.17 (d, J=8.5 Hz, 4H, benzylic), 4.53 (m, 1H), 4.33-4.22 (m, 2H), 4.18 (t, d, J=7.2 Hz, 1H), 3.28 (dd, J=13.8, 4.6 Hz, 1H), 3.08 (dd, J=13.8, 9.3 Hz, 1H). ¹³C-NMR (100 MHz, Acetone-d₆): δ 174.2 (C), 157.0 (C), 148.2 (C), 144.9 (C), 141.9 (C), 136.6 (C), 136.6 (C), 135.9 (C), 132.0 (CH), 131.7 (CH), 129.4 (CH), 128.9 (CH), 128.5 (CH), 127.9 (CH), 126.1 (CH), 120.7 (CH), 120.6 (CH), 80.5 (d, J=163.8 Hz, CH₂F), 70.7 (CH₂), 67.2 (CH₂), 56.4 (CH), 47.8 (CH), 37.4 (CH₂). ¹⁹F-NMR (376 MHz, Acetone-d₆): δ −215.0 (t, J=47.6 Hz). ³¹P-NMR (162 MHz, Acetone-d₆): δ −5.88. IR (KBr): 3035, 2956, 1723, 1499, 1250, 1212, 1017, 963, 740 cm⁻¹. HRMS calcd for C₃₉H₃₅NO₈FPNa (M+Na)⁺718.1982, found 718.1980.

### Example 7

PTP1B and TCPTP Labeling of Probe Compound 30b of the Invention

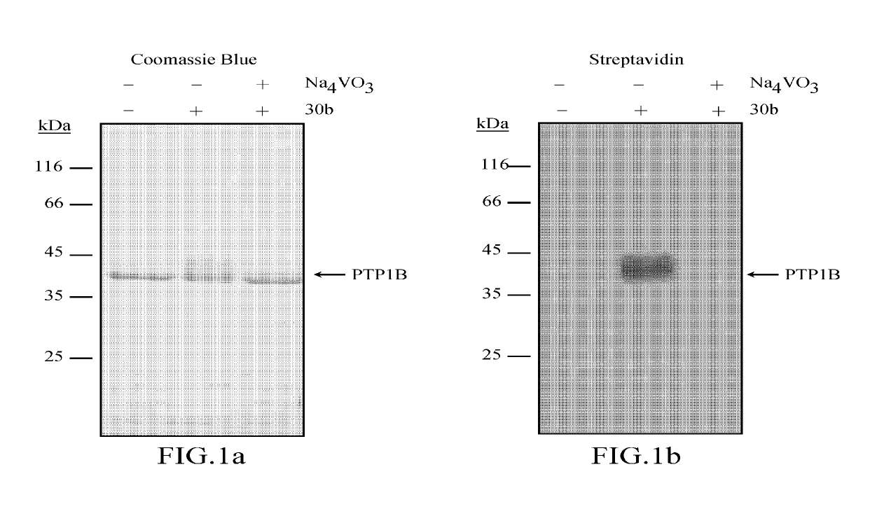

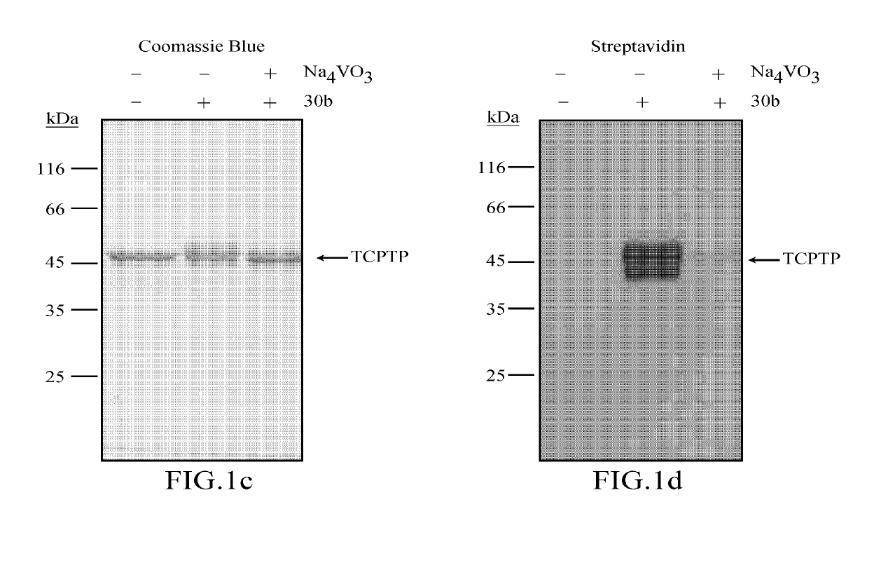

Referring to FIGS. 1a and 1b, FIG. 1a was stained with Coomassie blue, which showed the relative amount of loaded proteins. FIG. 1b was visualized by immunoblotting analysis (streptavidin) after transferring the reaction products onto a nitrocellulose membrane. Intense biotinylated protein bands were observed in PTP1B that was treated with probe compound 30b. In contrast, no biotinylated adduct was observed when Na₃VO₄, a phosphatase inhibitor, was present in the incubation mixture. Similar results were obtained when TCPTP was used as the labeling target (as shown in FIGS. 1c and 1d). Since the probe compounds themselves are also the substrates of the corresponding PTPs, the results clearly indicate that the newly developed latent trapping unit well mimics the phosphorotyrosine residue of the natural substrate. The results also indicate that the long tail containing the linker and the biotin reporter attached to the N-terminus of the tripeptide does not prevent the probe compound from entering the active site. Significantly, the labeling of PTPs with probe compound 30b was activity dependent. It should be noted that the benzylic fluoride moiety in probe compounds 30a-c showed reasonable stability in the labeling buffer. In the absence of PTPs, they underwent hydrolysis slowly and the purity remained greater than 90% even after 1 hr (as determined by HPLC).

### Example 8

Enzyme Specificity of Probe Compounds 30a-c of the Invention (1)

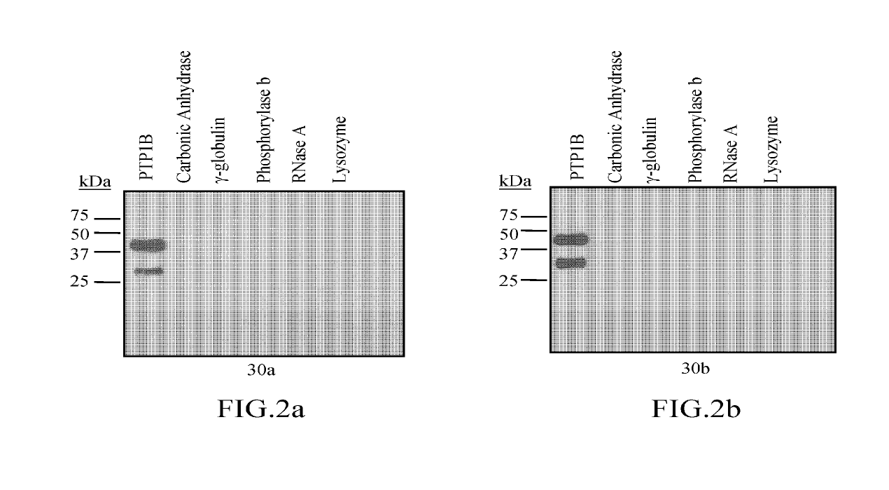

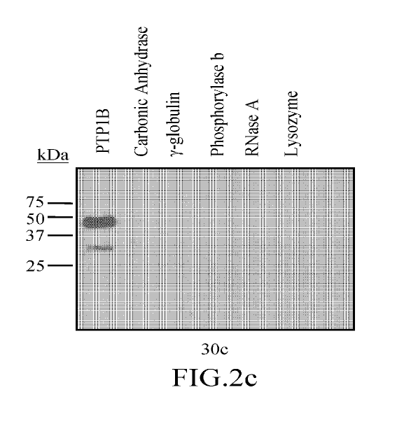

To further confirm the group specificity of the activity probe compounds, we compared the effect of probe compounds 30a-c on other proteins, including carbonic anhydrase, γ-globulin, phosphorylase b, RNase A, and lysozyme. The results obtained showed that probe compounds 30a-c did not label any of these proteins (as shown in FIGS. 2a to 2c).

### Example 9

Enzyme Specificity of Probe Compounds 30a-c of the Invention (2)

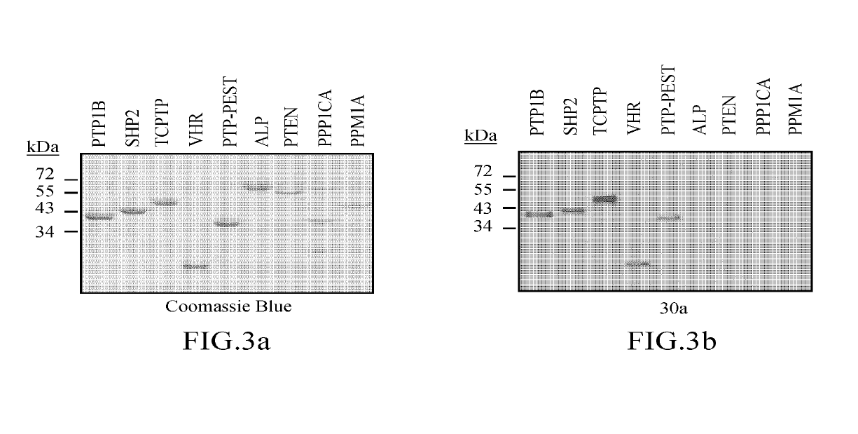

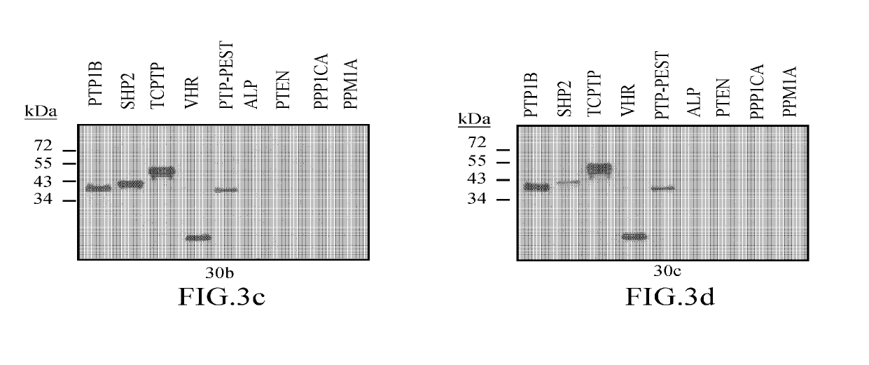

To test if probe compounds 30a-c could differentiate PTPs from other phosphatases, we conducted labeling experiments on nine phosphatases, including five PTPs, alkaline phosphatase (ALP), PTEN (dephosphorylating the phosphatidylinositol 3,4,5-trisphosphate), and two serine/threonine phosphatases (PPP1CA and PPM1A). The results showed that probe compounds 30a-c labeled all five PTPs and yet did not label any of the non-PTPs (as shown in FIGS. 3b to 3d), confirming that probe compounds 30a-c are indeed highly specific for PTPs.

### Example 10

Enzyme Selectivity of Probe Compounds 30a-c of the Invention

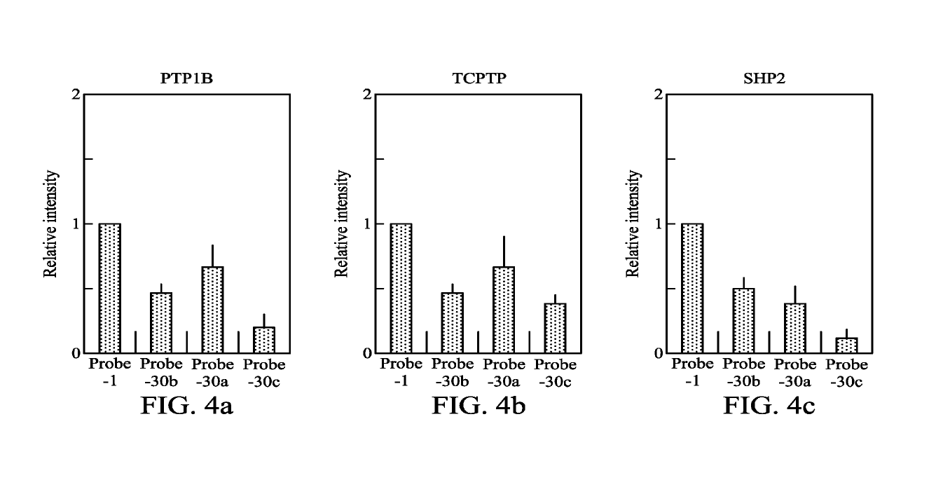

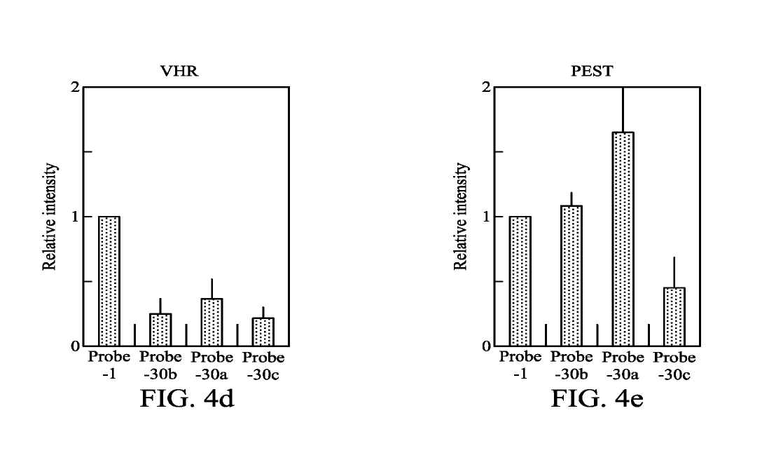

In order to further study how well probe compounds 30a-c could differentiate between the various PTPs, we compared the labeling intensities on five PTPs (PTP1B, SHP2, TCPTP, VHR, and PTPPEST) with those obtained from probe 1 (LCL2). When the intensities of the probe compounds 30a-c-labeled bands in each set of experiments were normalized relative to that of a probe 1-labeled band, we were able to establish a quantitative comparison of their labeling preference (as shown in FIGS. 4a to 4e). The result strongly suggests that the labeling intensity also reflects the trend of substrate specificities for these PTPs. Of the five PTPs tested, the substrate specificities of PTP1B, TCPTP, and SHP2 were better studied than those of VHR and PTP-PEST. The results for the former three PTPs showed that both PTP1B and TCPTP preferred probes with sequence Phe-pTyr-Leu over Glu-pTyr-Leu and Lys-pTyr-Leu, whereas SHP2 preferred Glu-pTyr-Leu and Phe-pTyr-Leu over Lys-pTyr-Leu, clearly supporting the observation that these probe compounds indeed labeled different PTPs with varying efficiency. The trend of substrate preference obtained from this study was similar to those determined by other methods. We have also compared the labeling intensity data for the other two PTPs, VHR (a dual-specificity phosphatase) and PTP-PEST (a classical PTP). The results obtained suggest that PTP-PEST showed a substrate preference similar to that of PTP1B and TCPTP, whereas VHR did not show much substrate preference. This provides evidence to support the concept that probe compounds with added amino acid residues flanking the latent trapping device were able to influence their target specificities.

### Example 11

Synthesis of Precursor Compound 10 of the Invention

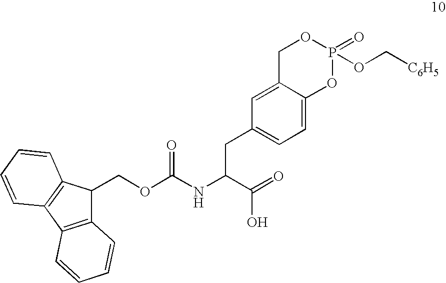

Synthesis Schemes:

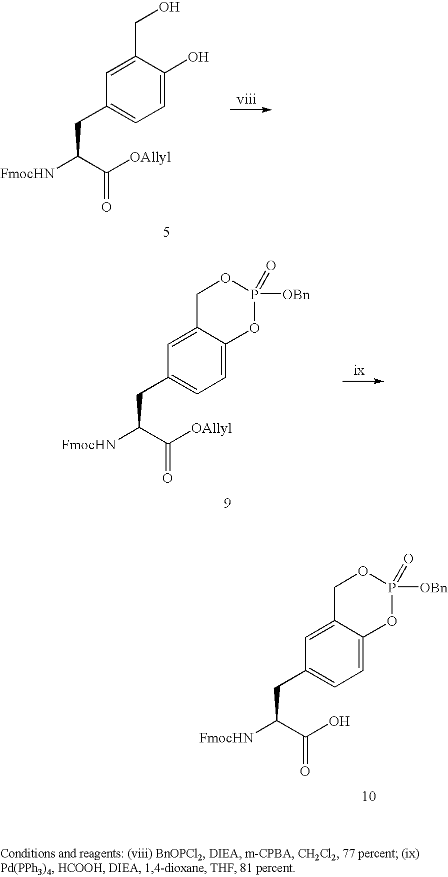

To an ice-cooled solution of compound 5 (500 mg, 1.06 mmol) and DIEA (872 L, 5.28 mmol) in 5.3 mL of anhydrous CH₂Cl₂ was added dropwise BnOPCl₂ (549 mg, 2.64 mmol). After stirring for 1 hr when no more starting material was observed by TLC, a solution of m-CPBA (784 mg, 3.18 mmol, 70wt %) in 2 mL of CH₂Cl₂ was added. The reaction mixture was stirred for 1 hr and then diluted with CH₂Cl₂. The reaction mixture was washed consecutively with 5% citric acid, 5% NaHCO₃, H₂O and brine. The organic layer was dried over anhydrous Na₂SO₄, filtered and concentrated. Compound 9 was obtained (510 mg, 77%) as colorless oil after silica gel column chromatography eluted with CH₂Cl₂/EtOAc (9/1).

To a solution of compound 9 (3.30 g, 5.27 mmol) in 26 mL of 1,4-dioxane/THF (1/1) was sequentially added HCOOH (597 μL, 15.8 mmol), DIEA (2.620 μL, 15.8 mmol), and Pd(PPh₃)₄ (183 mg, 0.158 mmol). After stirring for 14 hr the reaction mixture was dissolved in EtOAc and then washed consecutively with 5% citric acid, 5% NaHCO₃, H₂O and brine. The organic layer was dried over anhydrous Na₂SO₄, filtered, and concentrated. Compound 10 was obtained (2.50 g, 81%) as colorless foam after silica gel column chromatography eluted with 5-10% gradient MeOH in CH₂Cl₂.

¹H NMR (400 MHz, CD₃OD): 7.68 (d, J=7.2 Hz, 2H, aromatic), 7.55-7.42 (m, 2H, aromatic), 7.34-7.17 (m, 9H, aromatic), 7.13 (d, J=7.4 Hz, 1H, aromatic), 6.96 (s, 1H, aromatic), 6.80 (m, 1H, aromatic), 5.28-5.09 (m, 2H, benzylic), 5.08-4.97 (m, 2H, benzylic), 4.36 (m, 1H), 4.26-4.01 (m, 2H), 4.02 (s, 1H), 3.15 (d, J=12.4 Hz, 1H), 2.97-2.77 (m, 2H). ¹³C NMR (100 MHz, CD₃OD): 175.2 (C), 158.3 (C), 150.0 (C), 145.2 (C), 142.5 (C), 136.5 (C), 135.6 (C), 132.0 (CH), 130.0 (CH), 129.8 (CH), 129.3 (CH), 128.9 (CH), 128.2 (CH), 127.5 (CH), 126.3 (CH), 121.9 (C), 121.1 (CH), 119.4 (CH), 56.9 (CH), 48.3(CH), 71.5 (CH₂), 70.1 (CH₂), 67.9 (CH₂), 37.9 (CH₂). ³¹P NMR (162 MHz, CD₃OD): 8.70. IR (KBr): 3062, 3015, 2952, 1717, 1497, 1450, 1252, 1210, 1009, 741, 695 cm⁻¹. HRMS calcd for C₃₂H₂₇NO₈P (M⁻H)⁻ 584.1474, found 584.1475.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A probe compound for protein tyrosine phosphatase (PTP), as shown in Formula (I): 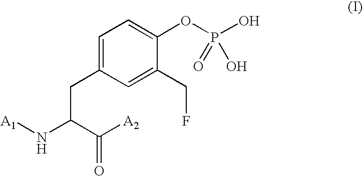
wherein
A₁ and A₂ represent amino acids.

2. The probe compound for protein tyrosine phosphatase (PTP) as claimed in claim 1, wherein the amino acids comprise leucine, phenylalanine, glutamic acid, lysine, alanine, arginine, aspartic acid, asparagine, citrulline, cysteine, cystine, glutamine, glycine, histidine, hydroxyproline, isoleucine, methionine, proline, serine, threonine, tryptophan, valine or a combination thereof.

3. The probe compound for protein tyrosine phosphatase (PTP) as claimed in claim 1, further comprising a linker connected to A₁ or A₂.

4. The probe compound for protein tyrosine phosphatase (PTP) as claimed in claim 3, wherein the linker comprises 2,2′-(ethylenedioxy)bis(ethylamine).

5. The probe compound for protein tyrosine phosphatase (PTP) as claimed in claim 3, further comprising a reporter group connected to the linker.

6. The probe compound for protein tyrosine phosphatase (PTP) as claimed in claim 5, wherein the reporter group comprises biotin.

7. A probe compound precursor for protein tyrosine phosphatase (PTP), as shown in Formula (II):

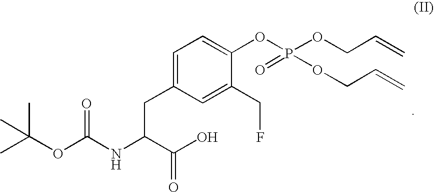

8. A probe compound precursor for protein tyrosine phosphatase (PTP), as shown in Formula (III): 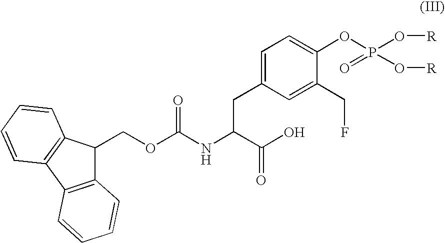
wherein
R represents —CH₂CH═CH₂ or —CH₂C₆H₅.

9. A probe compound for protein tyrosine phosphatase (PTP), as shown in Formula (IV): 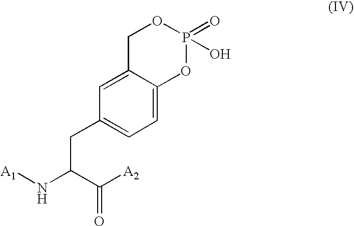
wherein
A₁ and A₂ represent amino acids.

10. The probe compound for protein tyrosine phosphatase (PTP) as claimed in claim 9, wherein the amino acids comprise leucine, phenylalanine, glutamic acid, lysine, alanine, arginine, aspartic acid, asparagine, citrulline, cysteine, cystine, glutamine, glycine, histidine, hydroxyproline, isoleucine, methionine, proline, serine, threonine, tryptophan, valine or a combination thereof.

11. The probe compound for protein tyrosine phosphatase (PTP) as claimed in claim 9, further comprising a linker connected to A₁ or A₂.

12. The probe compound for protein tyrosine phosphatase (PTP) as claimed in claim 11, wherein the linker comprises 2,2′-(ethylenedioxy)bis(ethylamine).

13. The probe compound for protein tyrosine phosphatase (PTP) as claimed in claim 11, further comprising a reporter group connected to the linker.

14. The probe compound for protein tyrosine phosphatase (PTP) as claimed in claim 13, wherein the reporter group comprises biotin.

15. A probe compound precursor for protein tyrosine phosphatase (PTP), as shown in Formula (V): [chemical structure]
